(19) United States
(12) Patent Application Publication
(10) Pub. No.: US 2008/0254139 A1
(43) Pub. Date: Oct. 16, 2008

(54) METHODS AND COMPOSITIONS TO TREAT OVARIAN CANCER

(76) Inventor: LEIGH H. FIRESTONE, Berkeley, CA (US)

Correspondence Address: LEIGH HUNT FIRESTONE, 7100 NORFOLK ROAD, BERKELEY, CA, 94705, US

(21) Appl. No.: 11/925,759

(22) Filed: Oct. 27, 2007

Related U.S. Application Data
(63) Continuation-in-part of application No. 11/708,231, filed on Feb. 20, 2007.
Application No. 11/708,231 is a continuation-in-part of application No. 11/865,023, filed on Sep. 30, 2007.
Application No. 11/865,023 is a continuation-in-part of application No. 11/876,963, filed on Oct. 23, 2007.
(60) Provisional application No. 60/775,913, filed on Feb. 22, 2006.
(60) Provisional application No. 60/947,412, filed on Jun. 30, 2007.

(30) Foreign Application Priority Data
Feb. 21, 2007 (US) 2007004332

(51) Int. Cl. A61K 35/407; A61K 35/12; A61K 35/22; A61P 35/00; A61K 35/38

(52) U.S. Cl. 424/553; 424/520; 424/551; 424/558

(57) ABSTRACT

The invention is a method of treating ovarian cancer by contacting a post-surgical site of an ovarian cancer patient with a composition or a sheet article comprising extracellular matrix to inhibit cancer cell attachment, promote wound healing after the surgery, reduce scar formation at the surgical site, regenerate lost tissue, and prevent tumor recurrence at the site or in the peritoneum generally.

### CROSS REFERENCE TO RELATED APPLICATIONS

The present application is a continuation in part to U.S. application Ser. No. 11/708,231 filed Feb. 20, 2007 which draws priority from U.S. provisional application 60/775,913 filed Feb. 22, 2006. The present application also claims priority from PCT application PCT/US2007/004332 filed Feb. 21, 2007. The present application draws priority from provisional application U.S. Ser. No. 60/947,412 filed Jun. 30, 2007. The present application is also a continuation in part of U.S. Ser. No. 11/865,023 filed Sep. 30, 2007. The present application is also a continuation in part of U.S. Ser. No. 11/876963 filed Oct. 23, 2007. The present application is a continuation in part of U.S. Ser. No. 11/935,753 filed concurrently Oct. 27, 2007. All related applications are specifically incorporated by reference in their entirety.

### FIELD OF THE INVENTION

The invention is to a method of treating ovarian cancer, and related compositions.

### BACKGROUND OF THE INVENTION

Carcinoma is the term for abnormally proliferating and poorly differentiating epithelial cells in mammals. Many types of carcinomas exist, including the very prevalent adenocarcinoma, which describes cancer of the epithelial surfaces of glandular tissues. Breast, colon, lung, thyroid, prostate, stomach, pancreatic, cervical, and ovarian cancers are all examples of tissues at which adenocarcinoma can form. In addition, carcinomas can originate in the bladder, uterus, kidney, lung, skin, and other tissues. This list is not exhaustive.

Types of normal healthy epithelium include simply squamous cells, simple cuboidal cells, simple columnar cells, stratified squamous cells, stratified cuboidal cells, pseudostratified columnar cells and transitional cells. Depending on the organ or tissue, the epithelial cells will take on their appropriate character within these categories.

Tumors are graded or staged depending on their level of differentiation and localization to the site of tumor origin. Staging refers to the condition of the tumor in the context of the surrounding tissues. So for example, Stage I ovarian tumors are confined to one or both ovaries. Stage II ovarian is ovarian cancer that has spread to pelvic organs, but not to abdominal organs. Stage III is ovarian cancer that has spread to abdominal organs. Stage IV is ovarian cancer that has spread outside to distant sites, for example the lung, brain, or lymph nodes in the neck. Within these stages there are subcategories that are identified based on tumor size, node involvement and metastatic status. Thus a tumor can be a IIA, which describes a tumor that has spread and attached to the uterus, where as a IIB tumor describes a tumor that has in addition spread to other pelvic tissues, but with no cancer cells in the ascites or peritoneum, and so on. In addition to tumor staging, epithelial tumors can also be graded. Grade refers to the character of the cells of the tumor. Grade 1 is the least malignant with well-differentiated cells, Grade 2 is intermediate with moderately differentiated cells, and Grade 3 is the most malignant with poorly differentiated cells. Low grade tumors grow more slowly and patients with them have a better prognosis for survival. Analogous staging and grading exists for all carcinomas and adenocarcinomas for every location in the body where the cancers can form.

Further continuing with the ovarian model, treatment of epithelial cancers usually involves a surgical first step especially if the tumor is Stage 1A or Stage 1B. Surgery can include hysterectomy (removal of the uterus), bilateral slpingectomy (removal of both fallopian tubes), bilateral oophorectomy (removal of both ovaries), and omenectomy (removal of part of the ornamentum, which is fatty tissue from the upper part of the abdominal cavity near the stomach and intestines). Pelvic and aortic lymph nodes may be sampled and the linings of the pelvis and abdominal cavities can be biopsied to determine if the cancer has spread. If the tumor is deemed a grade 1 or 2, meaning that the cells have some similarities to normal cells, surgery alone may be a cure for the patient. Even if the cancer has not spread, but turns out to be a grade 2 or 3 cell, then chemotherapy may be recommended after surgery. If the cancer is Stage II, then the standard protocol is to “debulk” the tumor in the pelvis as much as possible, which means to remove as much tumor tissue as can be located and safely removed. The protocol for Stage III and Stage IV are the same as for Stage II, with initial surgical treatment followed by chemotherapy. In some Stage III and Stage IV situations, the surgeon will administer chemotherapy directly to the tissues intraperitoneally, and patients have been found to survive longer with this aggressive approach. Follow-up surgery can include laparoscopy to determine if any tumors have regrown in the area of original surgery. Tumor recurrence can also be deduced from blood marker tests, palpation, positron emission tomography, CT scans, and magnetic resonance imaging (MRI).

A survey of the occurrence of carcinoma worldwide indicates that about 400,000 people die a year from carcinoma, and at least about 3 to 4 times that receive an initial diagnosis of carcinoma each year. Of these cancers, about 25,580 new cases of ovarian cancer were diagnosed in the US in 2004. About 16,090 deaths were reported in 2004 from ovarian cancer, which comes to an average of about 262 patients a week. About 4% of all diagnosed female cancers are ovarian cancer. In addition, females get other reproductive cancers such as uterine or follicular cancer, both localized to the peritoneal space and the reproductive organs located there.

It would be a great triumph for medicine and world health if a composition could be developed that lowered the risk of death by cancer in patients diagnosed with ovarian carcinoma and other female reproductive cancers localized to the peritoneum by providing an effective treatment that significantly prolongs their lives.

### SUMMARY OF THE INVENTION

The invention is a method of treating a patient having ovarian carcinoma comprising identifying an ovarian carcinoma in a female patient, surgically removing said carcinoma from said patient, and contacting a peritoneum and tissues proximal to said removed ovarian carcinoma with a therapeutically effective amount of extracellular matrix.

The invention is also a method of treating a patient having a reproductive carcinoma comprising identifying a reproductive carcinoma in a female patient, surgically removing said carcinoma from said patient, and contacting a peritoneum and tissues proximal to said removed carcinoma with a therapeutically effective amount of extracellular matrix.

The invention is also a composition for contacting a peritoneum of a female having a reproductive carcinoma comprising extracellular matrix in a liquid, semi-solid, or solid form, and further comprising an additional molecule or cell to prevent recurrence of said carcinoma.

The invention is a method of treating a patient having ovarian carcinoma comprising: a) identifying an ovarian carcinoma in a female patient, b) surgically removing said carcinoma from said patient, and c) contacting a peritoneum and tissues proximal to said removed ovarian carcinoma with a therapeutically effective amount of extracellular matrix. The method can further comprise surgically closing said patient. The method can also comprise monitoring said patient for wound healing. The method can further comprise monitoring said patient for recurrence of said carcinoma.

The extracellular matrix can be in a form selected from the group consisting of a solid, a semi-solid, and a liquid. The extracellular matrix can be mammalian. The extracellular matrix can be semi-solid or liquid in a concentration greater than about 0.001 ng/ml. The extracellular matrix can be porcine. The extracellular matrix can be bovine. The extracellar matrix can be human. The extracellular matrix can be small intestine submucosa. The extracellular matrix can be liver basement membrane. The extracellular matrix can be urinary bladder submucosa. The extracellular matrix can be stomach submucosa. The extracellular matrix can be a particulate. The extracellular matrix can be an emulsion. The extracellular matrix can be a gel. The extracellular matrix can be a sheet or laminate of two or more sheets.

The invention further includes a method of treating a female patient having a reproductive carcinoma located in a peritoneum of said patient comprising, a) identifying said reproductive carcinoma in said female patient, b) surgically removing said carcinoma from said patient, and c) contacting a peritoneum and tissues proximal to said removed carcinoma with a therapeutically effective amount of extracellular matrix.

The carcinoma can be selected from the group consisting of ovarian. The carcinoma can be uterine. The carcinoma can be follicular.
The method can further comprise monitoring said patient for wound healing. The method can further include monitoring the patient for scar formation. The method can further include monitoring the patient for tissue regeneration. The method can further comprise monitoring the patient for recurrence of the carcinoma.

The extracellular matrix can be in a form selected from the group consisting of a solid, a semi-solid, and a liquid. The extracellular matrix can be mammalian. The extracellular matrix can be semi-solid or liquid in a concentration greater than about 0.001 ng/ml. The extracellular matrix can be porcine. The extracellular matrix can be bovine. The extracellar matrix can be human. The extracellular matrix can be small intestine submucosa. The extracellular matrix can be liver basement membrane. The extracellular matrix can be urinary bladder submucosa. The extracellular matrix can be stomach submucosa. The extracellular matrix can be a particulate. The extracellular matrix can be an emulsion. The extracellular matrix can be a gel. The extracellular matrix can be a sheet or laminate of two or more sheets.

The invention also includes a method comprising detecting an ovarian tumor in a patient a)accessing a peritoneal space comprising said tumor using a minimally invasive surgical device, and b) lavaging said peritoneal space comprising said tumor comprising said tumor with a composition comprising extracellular matrix.

The extracellular matrix can be in a form selected from the group consisting of a solid, a semi-solid, and a liquid. The extracellular matrix can be mammalian. The extracellular matrix can be semi-solid or liquid in a concentration greater than about 0.001 ng/ml. The extracellular matrix can be porcine. The extracellular matrix can be bovine. The extracellar matrix can be human. The extracellular matrix can be small intestine submucosa. The extracellular matrix can be liver basement membrane. The extracellular matrix can be urinary bladder submucosa. The extracellular matrix can be stomach submucosa. The extracellular matrix can be a particulate. The extracellular matrix can be an emulsion. The extracellular matrix can be a gel. The extracellular matrix can be a sheet or laminate of two or more sheets.

### DETAILED DESCRIPTION OF THE INVENTION

The invention is to methods for treating female patients having ovarian carcinoma or other reproductive carcinoma located in the peritoneum, such as follicular of uterine cancer. These cancers manifest abnormally proliferating epithelial cells in epithelial tissue. Abnormally proliferating cells form carcinomas which are tumors located at an epithelial surface of the particular organ or tissue of origin. As the carcinoma takes hold at a particular site a single abnormally proliferating cancer cell can become a group of such cells or a tumor or lesion. A tumor of such cells can be recognized, graded and staged. Stages typically include Stage IA, IB, IIA, IIB, IIC, III, and IV. Tumor cells are graded from grade 1 (least malignant), grade 2 (intermediate), and grade 3 (the most malignant with poorly differentiated cells). Metastasis begins when cells at the original site break away from the original aggregate of cells and move through the body to attach and proliferate elsewhere. In the beginning the abnormally proliferating cells can be characterized as hyperplastic and somewhat resembling of normal epithelial cells in the region. As the cells progress to carcinoma grade cells and a worse stage of tumor they become malignant, poorly differentiated, and neoplastic, resembling less and less the original epithelial cells of the primary organ. Carcinomas can develop in any epithelial tissue in the body, and when that tissue is a glandular tissue, the cancer is an adenocarcinoma. For the purposes of this invention, female reproductive cancers include ovarian, follicular, uterine, or cervical cancer, or any cancer in any tissue of the female peritoneum in the region of these reproductive organs.

The method of treatment of the invention is to identify the reproductive carcinoma in the female patient. Where, for example, the carcinoma is ovarian carcinoma, blood markers, CT scans, palpation, localized pain, magnetic resonance imaging and other detection techniques can be used to identify the presence of the carcinoma. Some reproductive cancers of the ovaries or follicles, for example, are particularly elusive of identification, and may only be identified after surgery by histology. Generally, however, the carcinoma will first be identified and then surgery will be performed to remove the cancerous tumor or tumors. During surgery, a surgeon will visually identify all tumors possible and remove these tumors from the body. Depending on the stage of carcinoma in the patient, some or all of the visible tumors will be removed.

Thereafter, before surgical closure, the method of the invention dictates contacting all tissues possible in the peritoneum with a composition comprising extracellular matrix. The sites of excised tissue are particularly important to contact or coat with the composition. If possible, as many tissues as possible in the peritoneum are contacted or coated with the extracellular matrix composition. It is particularly important to contact all tissues in the peritoneum if the cancer is a stage II or worse, and if the carcinoma cells are poorly differentiated. This is because the cancer may have spread to these areas without having become evident enough to be seen by the naked eye.

The composition of the invention is extracellular matrix in a solid form (such as a sheet or dry particulate), or as a liquid or semi-solid form, such as a liquid or emulsion. The solid form is placed in contact with the tissues where it is desirable to prevent cancer growth on the surface of the tissue. Thus, a particulate can be dusted or sprayed at the site. A piece of extracellular matrix can be placed at the site, or otherwise affixed to a surgically treated area. The solid extracellular matrixes can be a sheet, a particulate, a small piece, patch, strip, pellet, plug, strand, weave, or any other form of solid extracellular matrix suited to the task of contacting the abnormal cells or the tissue at the resected tumor site, and neighboring tissues in vivo. Preferred forms are sheets or particulate. Particulate can be made from drying sheets and breaking them up in to fine powder that can be stored, reconstituted, used as is, or delivered in a particular mode, for example by spray or dusting.

The composition can also be extracellular matrix in liquid or semi-solid form (e.g. an injectable solution, a gel or an emulsion) at concentrations that have not before been created or used. In one aspect the concentration of liquid or semi-solid extracellular matrix is greater than about 0.001 mg/ml. Accordingly, the concentration of this composition can be any of the following concentrations and those concentration in between these numbers including about 0.001 mg/ml, 0.002 mg/ml, 0.003 mg/ml, 0.004 mg/ml, 0.005 mg/ml, 0.006 mg/ml, 0.007 mg/ml, 0.008 mg/ml, 0.009 mg/ml, 0.01 mg/ml, 0.02 mg/ml, 0.03 mg/ml, 0.04 mg/ml, 0.05 mg/ml, 0.06 mg/ml, 0.07 mg/ml, 0.08 mg/ml, 0.09 mg/ml, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.5 mg/ml, 2.0 mg/ml, 2.5 mg/ml, 3.0 mg/ml, 3.5 mg/ml, 4.0 mg/ml, 4.5 mg/ml, 5.0 mg/ml, 5.5 mg/ml, 6.0 mg/ml, 6.5 mg/ml, 7.0 mg/ml, 7.5 mg/ml, 8.0 mg/ml, 8.5 mg/ml, 9.0 mg/ml, 9.5 mg/ml, 0.0 mg/ml, 12 mg/ml, 15 mg/ml, 17 mg/ml, 20 mg/ml, 25 mg/ml, 28 mg/ml, 30 mg/ml, 33 g/ml, 35 mg/ml, 38 mg/ml, 40 mg/ml, 42 mg/ml, 45 mg/ml, 48 mg/ml, 50 mg/m, 55 mg/ml, 60 mg/ml, 65 mg/ml, 70 mg/ml, 75 mg/ml, 80 mg/ml, 85 mg/ml, 90 mg/ml, 95 mg/ml, 100 mg/ml, 105 mg/ml, 110 mg/ml, 115 mg/ml, 120 mg/ml, 125 mg/ml, 130 mg/ml, 135 mg/ml, 140 mg/ml, 145 mg/ml, 150 mg/ml, 155 mg/ml, 160 mg/ml, 165 g/ml, 170 mg/ml, 175 mg/ml, 180 mg/ml, 185 mg/ml, 190 mg/ml, 195 mg/ml, and 200 g/ml.

The solid, semi-solid, or liquid forms of extracellular matrix can further comprise one or more molecules or moieties to help inhibit the cancer or prevent cancer recurrence. Thus an additional molecule can be a protein, a small organic molecule, or a drug, for example. Additionally, the composition can also comprise a cell, such as a stem cell. Such a molecule or additive can be categorized as, for example, an anti-cancer agent, an anti-proliferative agent, an antibiotic, a wound healer, a chemotherapeutic agent, a chemotactic agent, e.g. for endogenous stem cells or active protein molecules that would work to the patient's advantage if recruited to the site.

The extracellular matrix for the compositions and methods is from a mammalian source and can be any extracellular matrix from any tissue in a mammal. Mammals can include humans, horses, monkeys, cows, pigs, sheep, dogs, rabbits, rodents, and generally any otherwise healthy mammal. The extracellular matrices can be from any mammalian tissue having an extracellular matrix, particularly matrices that support soft tissue. The extracellular matrices can be from, for example, small intestine, liver, urinary bladder, stomach, pancreas, placenta, large intestine, heart, lung, kidney, and in general any tissue in the mammalian body. Fetal extracellular matrices can be used from any fetal organ of any mammal. It is also possible that other animals, including vertebrates, including vertebrates such as fish and birds may provide extracellular matrix of sufficient quality for use in the compositions.

Extracellular matrix is a generic term for the proteinous material that exists outside of cells in tissues, supporting cells and cell-protein interactions, among many other functions. Extracellular matrix from epithelial tissues typically has a basement membrane layer, a submucosal layer, and a mucosal layer. Some tissues have an interstitial layer, lamina propria, tunica propria and other layers of matrix. Many of these layers are distinct for particular tissues, and some layers have synonymous terms in the same or different tissues. The invention contemplates any and all of these layers or divisions of matrix, either all together, or separately, or in various combinations to form the compositions of the extracellular matrix. It is generally believed that the submucosal layer is the most active and important of the extracellular matrix layers, and as such, preferred compositions and methods include at least a submucosa in the composition. Submucosa as derived from some mammalian tissues is described in U.S. Pat. No. 4,902,508, U.S. Pat. No. 5,281,422, U.S. Pat. No. 5,281,422, U.S. Pat. No. 5,275,826, U.S. Pat. No. 5,554,389, and other related US patents. Liquid extracts of these matrices and how to make them are described in U.S. Pat. No. 6,375,989 and U.S. Pat. No. 6,579,538, describing how to make liquid and semi-solid extracellular matrix compositions. All of these patents and any related commonly owned patents and patent applications with supportive disclosure are hereby incorporated by reference in their entirety. Generally to make the extracts of matrices, sheets are prepared from the tissues and lyophilized, and then broken up into a fine powder that can be reconstituted in saline or other suitable buffer at a desired concentration.

If the extracellular matrix used is small intestine submucosa (SIS), or tela submucosa which is a multi-laminate structure comprising tunica submucosa, lamina muscularis mucosa, and the stratum compactum, the SIS can be made as described in WO 98/22158, which is the published application of PCT/US97/14855. Other mucosas can also be used, including liver tissue as described in WO 98/25637 (PCT/US97/22727), gastric mucosa as describe in WO 98/26291 (PCT/US9722729), stomach submucosa as described in WO 98/25636 (PCT/US97/23010), and from urinary bladder mucosa as described in U.S. Pat. No. 5,554,389. Other related patents also containing supporting disclosure for this invention include U.S. Pat. No. 4,902,508, U.S. Pat. No. 4,956,178, U.S. Pat. No. 5,281,422, U.S. Pat. No. 6,099,567, U.S. Pat. No. 6,379,710, U.S. Pat. No. 4,361,552, U.S. Pat. No. 6,576,618, and WO 00302165. Additionally, optimizing the condition of these materials for placement in humans is described in U.S. Pat. No. 6,666,892 and U.S. Pat Pub 2006/147433. Powder forms of ECM can be formed as described in U.S. Pat. No. 5,275,826, WO 96/32146, WO 98/25636, and fluidized forms of ECM can be formed as described in U.S. Pat. No. 5,275,826. Sheet constructs can be formed as described in U.S. Pat. No. 5,127,903 and WO 96/32146. All these references are incorporated by reference in their entirety.

The tela submucosa can be fluidized, emulsified, as dry particles, or sheets, or some combination of these forms. The article or material placed in the patient can be coiled, helical, spring-like, randomized, branched, sheet-like, tubular, spherical, fragmented, fluidized, comminuted, liquefied, suspended, gel-like, injectable, powdered, ground, sheared, and solid material shape. The tela submucosa can be placed in the patient alone, or in combination with one or more bioactive agents such as physiologically compatible minerals, growth factors, antibiotics, chemotherapeutic agents, antigen, antibodies, enzymes, and hormones.

Additives can be mixed into the extracellular matrix. Additives can include such molecules as immunotherapeutic molecules, cells, anticancer agents, nucleic acids, peptides, polypeptides and proteins. A particularly preferred embodiment of the composition is an extracellular matrix in liquid, emulsion or dry powder form having one or more anti-cancer agents directed to a female reproductive cancer. Such agent can be, for example, a hormone, antibody, antigen, chemotherapeutic agent, and a small molecule inhibitor specific for ovarian, uterine, or follicular cancer.

One method of the invention is inhibiting abnormal proliferation of an epithelial cell in epithelial tissue in a patient afflicted with an epithelial cell proliferation disorder of a female reproductive tissue. The epithelial cell proliferation disorder of the female reproductive tissue will typically be carcinoma, pre-carcinoma, neoplasia, pre-cancer, cancer, epithelial cell cancer, or any known synonyms or near synonyms for the same. The cells can be hyperplastic, displastic, pre-malignant, malignant, moderately differentiated, poorly differentiated, or the like. Abnormal proliferation is manifest in cells that proliferate. Normal healthy cells do not proliferate once they have terminally differentiated into a tissue type. Cancer cells characteristically manifest abnormal proliferation and therefore grow in clumps that become visible tumors. The cell or cells will be located at a tissue of origin, thus an epithelial tissue, and the cell or cells will generally be proliferating abnormally in the context of an endogenous epithelial extracellular matrix that surrounds and supports the epithelial cells of the region. The affected region can be ovary, follicle, uterine, or cervix, or other reproductive tissue found in the peritoneum of female patients.

In practicing the method, the abnormally proliferating epithelial cell of reproductive origin is contacted with a composition of a soft tissue mammalian extracellular matrix. After a passage of some time to allow the composition to have an effect, inhibition of cell proliferation of this cell or cells is detected. It is anticipated that fewer abnormally proliferating cells will be detected than previously existed at the site before contact with the composition, or that the abnormal appearance of a particular cell will have altered to have it appear normal or nearly normal again.

Inhibition of cell proliferation in the epithelial tissue in the patient can be manifest by an absence of such abnormally proliferating cells after a period of time, a reduction in quantity of such abnormally proliferating cells, or an improvement in grade (such as greater cell differentiation) in the cells. The most preferred result is the complete absence of such abnormally proliferating cells, and thus that the abnormally proliferating cells are undetectable or no longer detectable at the site. Generally, absence of recurrence of the cancer at the surgical site is considered a successful result to the treatment.

Another method of the invention is preventing recurrence of an epithelial tumor in a patient. The method is practiced by removing at least some of the epithelial tumor from a patient forming a resected tumor site. After that, the resected tumor site is contacted with a composition comprising mammalian soft tissue extracellular matrix. The site is closed and enough time is allowed for the site to heal. At some time point later, the area is monitored (preferably non-invasively) for signs of regrowth of the tumor. A preferred result is absence of any regrowth of the tumor.

The extracellular matrix used to contact the tumor site can be any form of extracellular matrix, but is preferably a form that provides maximal contact with the site, such as a particulate, semi-solid, or liquid extracellular matrix. Particularly in the case of the creation of a tumor cavity with the excision of a tumor, extracellular matrix is used to fill the cavity. Due to its wound healing capabilities, the extracellular matrix can heal the healthy resected tissue, which inevitably gets excised while trying to excise a tumor. The extracellular matrix also serves to redirect growth and differentiation of any of the remaining malignant tissue, to reprogram its course to become normal tissue again.

The invention is an article or composition comprising mammalian extracellular matrix (ECM) for placing in the space remaining after a tumor excision in epithelial tissue in which both a tumor and tissue next to the tumor are excised. The article can be a sheet or several sheets of ECM. The sheets can be placed in the excised tumor space, crumpled into balls, or wads and placed in the space in the breast, or rolled loosely or tightly and configured to fit into the space left after the tissue and tumor have been excised. The composition is particulate ECM or emulsion or gel ECM.

Accordingly, the particulate, emulsion or gel compositions can comprise ECM and other materials as well. Optimally, the composition comprises only ECM, and the ECM been processed so as to retain key growth factors and other molecules and proteins so that when the ECM is placed in the body, the ECM remodels to become the host tissue with which it is in contact. Other materials added to the ECM could be, for example, a therapeutic agent, a drug, added proteins or added cells.

Natural ECM materials suitable for use with the present invention include mammalian small intestine submucosa (SIS), stomach submucosa (SS), urinary bladder submucosa (UBS), dermis, or liver basement membranes (LBM) derived from sheep, bovine, porcine or any suitable mammal. Small intestine submucosa (SIS) is described in U.S. Pat. Nos. 4,902,508, 4,956,178, and 5,275,826; urinary bladder submucosa (UBS) is described in U.S. Pat. No. 5,554,389, stomach submucosa (SS) is described in U.S. Pat. No. 6,099,567, and liver submucosa (LS) or liver basement membrane (LBM) is described in U.S. Pat. No. 6,379,710. See also U.S. Pat. No. 5,554, 389, U.S. Pat. No. 4,902,508, and U.S. Pat. No. 5,281,422. All of these patents and any related commonly owned patents and patent applications with supportive disclosure are hereby incorporated by reference in their entirety.

Although these particularly named extracellular matrices are known and have been isolated and used, there may be other mammalian tissues from which extracellular matrix can be isolated and prepared and as such would be suitable for the purposes of the invention. Extracellular matrix-like materials are also generally described in the article “From Cell-ECM Interactions to Tissue Engineering”, Rosso et al, Journal of Cellular Physiology 199, 174-180 (2004). Enamel matrices, which are the extracellular matrix in the tissue around forming teeth, are described in U.S. Pat. No. 7,033,611. The disclosures of all references cited herein are incorporated in their entirety by reference. Extracellular matrices from these tissues have been isolated, processed to retain key growth factors and structural molecules, and dried to become solid materials (sheets and particulates). Particulate forms can be rehydrated in a suitable buffer to become fluidized or emulsion or gel forms. Presently, these extracellular matrix articles and compositions are used for tissue grafting, wound healing, and tissue regenerative purposes.

The invention proposes use of these ECM articles and compositions and materials and forms for placement in the space remaining after a tumor excision as a surgical procedure for the purpose of reducing a likelihood of tumor recurrence in the at the site of tumor excision. The ECM articles and compositions and materials and forms are also proposed in order to regenerate the tissue lost from the organ or tissue during the tumor excision. In addition, the ECM articles and compositions and materials and forms are also proposed in order to reduce localized scarring that can result from the surgical wound created by the surgical procedure.

Methods of use of the articles and compositions of the invention are also contemplated, for example as part of a routine tumor removal or debulking procedure to excise an epithelial tumor or carcinoma, and replace the space in the organ or tissue with mammalian ECM in a sheet form (i.e. as an article) or as a particulate or emulsion or gel form (i.e. as a composition). Surgical excision of the tumor, and placement of the ECM in the space in the organ or tissue after the excision is followed by closing the surgical wound with the ECM article or composition in the breast. Tissue regeneration or wound healing occurs within about 3 to 6 months post surgery.

Mammalian tissue sources are in general any tissue having an extracellular matrix that can be isolated from a mammal and de-cellularized. Thus for example, most mammalian organs are tissue sources. The tissue sources can be for example any mammalian tissue, including but not limited to the small intestine, large intestine, stomach, lung, liver, kidney, pancreas, placenta, heart, bladder, prostate, tissue surrounding growing tooth enamel, tissue surrounding growing bone, and any fetal tissue from any mammalian organ. The decellularization process is important as the material needs to be without its native cells, but the process of the removing the cells need not be so stringent as to remove key active growth factors that contribute to the material's usefulness in the human body. Processes for isolated extracellular matrix from tissues are known in the art, as are processes of decellularizing these matrices.

Extracellular matrix can be obtained from the tissues of mammals by processes such as described in U.S. Pat. No. 5,554, 389, U.S. Pat. No. 4,902,508, and U.S. Pat. No. 5,281,422. For example, the urinary bladder submucosa is an extracellular matrix that has the tunica mucosa (which includes the transitional epithelial layer and the tunica propria), a submucosal layer, 3 layers of muscularis, and the adventitia (a loose connective tissue layer). This general configuration is true also for small intestine submucosa (SIS) and stomach submucosa (SS). Obtaining enamel matrices is described in U.S. Pat. No. 7,033,611. Enamel matrix is extracellular matrix existing near forming teeth.

Natural ECM materials include mammalian small intestine submucosa (SIS), stomach submucosa (SS), urinary bladder submucosa (UBS), dermis, or liver basement membranes (LBM) derived from sheep, bovine, porcine or any suitable mammal. Small intestine submucosa (SIS) is described in U.S. Pat. Nos. 4,902,508, 4,956,178 and 5,275,826; urinary bladder submucosa (UBS) is described in U.S. Pat. No. 5,554,389, stomach submucosa (SS) is described in U.S. Pat. No. 6,099,567, and liver submucosa (LS) or liver basement membrane (LBM) is described in U.S. Pat. No. 6,379,710. In the preparation process, native extracellular matrices are prepared so that their bioactivity is preserved, including many cellular and transcriptional and translational event. Assays for determining these activities are standard in the art.

Many of these ECM compositions are generally comprised of the same tissue layers and are prepared by the same method, the difference being that of the starting material (i.e. from one organ versus another). The matrices are generally decellularized in order to render them non-immunogenic, a process that needs to also retain some function of key proteins, such as some growth factors. Specific procedural steps are further detailed in the patents referenced above.

Examples of a typical epithelium having a basement membrane include tissues that have an epithelium such as the skin, intestine, urinary bladder, esophagus, stomach, cornea, and liver. The epithelial basement membrane may be in the form of a thin sheet of extracellular material contiguous with the basilar aspect of epithelial cells. Sheets of aggregated epithelial cells of similar type form an epithelium. Epithelial cells and their associated epithelial basement membrane may be positioned on the luminal portion of the tunica mucosa and constitute the internal surface of tubular and hollow organs and tissues of the body. Connective tissues and the submucosa, for example, are positioned on the abluminal or deep side of the basement membrane, and can include for example the submucosa of the intestine (SIS) and urinary bladder (UBS), and the dermis and subcutaneous tissues of the skin. Typically the material is rinsed with saline and optionally stored in a frozen hydrated state until used.

In addition to employing sheet ECMs to form the articles of the present invention, the ECM material may be fluidized or emulsified. Fluidized UBS, for example, can be prepared in a manner similar to the preparation of fluidized intestinal submucosa, as described in U.S. Pat. No. 5,275,826. The UBS is comminuted by tearing, cutting, grinding, shearing or the like. Grinding the UBS in a frozen or freeze-dried state is preferred although good results can be obtained as well by subjecting a suspension of submucosa pieces to treatment in a high speed (high shear) blender and dewatering, if necessary, by centrifuging and decanting excess water. Additionally, the comminuted fluidized tissue can be solubilized by enzymatic digestion of the bladder submucosa with a protease, such as trypsin or pepsin, or other appropriate enzymes for a period of time sufficient to solubilize said tissue and form a substantially homogeneous solution.

Other examples of ECM material suitable for use with the present invention include but are not limited to dermal extracellular matrix material, subcutaneous extracellular matrix material, large intestine extracellular matrix material, placental extracellular matrix material, ornamentum extracellular matrix material, heart extracellular matrix material, and lung extracellular matrix material, may be used, derived and preserved similarly as described herein for the SIS, SS, LBM, and UBM materials. Other organ tissue sources of basement membrane for use in accordance with this invention include spleen, lymph nodes, salivary glands, prostate, pancreas and other secreting glands. In general, any tissue of a mammal that has an extracellular matrix can be used for developing an extracellular matrix component of the invention.

Other tissues such as the liver and pancreas have a basement membrane that does not demonstrate the kind of tensile strength of the tissues defined as submucosa. However, other useful properties may be opportunistically employed from the extracellular matrices of such tissues as the liver, pancreas, placenta and lung tissues which have either basement membrane for extracellular matrix or interstitial membrane (as with the lung). These softer matrices support cells such as those in the organs from which the matrices are derived. Thus, certain benefits are to be found in using the extracellular matrices of these tissues, especially in combination with other such matrices like SIS and SS that may be stronger and which offer their particular advantages. Accordingly, any of these mammalian matrices can be used with potential effectiveness in certain tissues or organs having carcinoma. Accordingly, the liver, lung, and pancreatic extracellular matrices may be quite suitable for generating some of the sheets, strips or pieces of the articles of the invention, or particulates or gels and may be used as such, such as, for example articles or compositions to be placed in organs or tissues after tumor removal such as liver tumors, lung tumors, and pancreatic tumors.

A method of the invention provides a composition comprising mammalian extracellular matrix from any source, identifying a reproductive epithelial tumor in a female mammal, the tumor comprising abnormally proliferating epithelial cells, disrupting one or more cells of the tumor or the tissue surrounding the tumor, to form a disrupted tumor site, and contacting the disrupted tumor site with a therapeutically effective amount of the extracellular matrix composition. Disruption of the tumor cells or the tissues surrounding the tumor is accomplished so that a wound area is created at the site. Disruption can be accomplished by scraping, poking, cutting, or otherwise touching the cells of the tumor and surrounding tissue. Upon contact with the extracellular matrix this damaged tissue of epithelial origin will begin to heal, thus altering what would have been an inevitable growth of cancerous tissue overtaking the healthy tissue. Contact of the region with healthy extracellular matrix from any source will redirect the area to heal from both the recent disruption, and the carcinoma.

The tumor targeted by this method is preferably stage II or worse, including stage IIA, IIB, IIC, stage IIIA, IIIB, IIIC, and stage IV. Often tumors at stage II or worse can not be completely resected with clean margins. Therefore disrupting the tumor, and perhaps removing most of it, will allow the composition of extracellular matrix to come into the region as a therapeutic and heal the disrupted, damaged tissue. The disruption of the tumor can involve not removing any tumor, but rather scrapping or cutting the tumor in situ, providing an opportunity for the extracellular matrix composition to contact the tumor cells more completely. Generally, surgeons do not favor disrupting tumor cells without removing them for fear that a disrupted tumor cell will metastasize to another region of the body, but in the case of this method, all tumor cells are covered or coated with extracellular matrix before closing the site by using liquid or semi-solid extracellular matrix to encase the dislodged tumor and prevent the migration of its cells to another location in the body.

The extracellular matrix can be from a soft tissue mammalian extracellular matrix, and is preferably of epithelial tissue origin. The epithelial tumor is growing in contact with epithelial tissue of tumor origin. By disrupting both the tumor and the surrounding epithelial tissue, upon placement at the site of extracellular matrix composition (e.g. a liquid, semi-solid, or solid extracellular matrix) the new exogenous extracellular matrix can begin to heal the epithelial tissue and generate healthy tissue at the site.

A method of the invention is also a method of healing a wound in epithelial tissue at a site of excision of an epithelial tumor. This method is practiced by excising at least a part of an epithelial tumor from a mammal, and at least some of the surrounding epithelial tissue forming a tumor excision site, contacting the tumor excision site with a therapeutically effective amount of a soft tissue mammalian extracellular matrix and monitoring the site for healing of the surrounding epithelial tissue. The monitoring can be done non-invasively, e.g. by a visualization means or using blood markers. The extracellular matrix of the composition can be liquid, semi-solid, or solid matrix. The solid matrix can be a particulate, or a sheet, or a laminate of sheets.

Another method of the invention is a method of inhibiting proliferation of abnormally proliferating epithelial cells in epithelium of a reproductive organ in a female mammal comprising locating a lesion of abnormally proliferating epithelial cells in epithelial tissue of a reproductive organ, excising at least some of these cells in the lesion and at least some of the epithelial tissue forming a site of tissue disturbance. The next step is contacting the site of tissue disturbance with a therapeutically effective amount of a soft tissue extracellular matrix and monitoring said site for inhibition of proliferation of the abnormally proliferating epithelial cells. In this method, the composition comprising extracellular matrix contacts the region of tissue disturbance and inhibits the abnormally proliferating epithelial cells. The inhibition can be monitored non-invasively by visualizing the site periodically after the initial procedure, or measuring blood markers that indicate the particular carcinoma being treated.

It has not been previously appreciated that recruitment of endogenous stem cells to a site of abnormally proliferating cells in mammalian tissue results in inhibition of the abnormal proliferation of the cells. It is known that endogenous stem cells are recruited to a site of tissue remodeling that is directed with the placement of exogenous extracellular matrix at a site. It has not been before appreciated that placement of exogenous mammalian extracellular matrix at a site of malignancy will result in stem cell participation in the inhibition of the malignancy. Thus the invention includes a method of recruiting endogenous stem cells to a site of abnormally proliferating cells in mammalian tissue by contacting an abnormally proliferating cell in a tissue type of a mammal, with a composition comprising exogenous mammalian extracellular matrix (extracellular matrix from another mammal) thereby recruiting one or more endogenous stem cells to the site. Upon recruitment to the site, the stem cells begin to facilitate remodeling of the damaged and diseased tissue, which has a great effect on the course of the malignancy. Malignant cells become less malignant, and eventually become normal cells and normal tissue as a result of the intervention. The stem cells that are recruited are probably adult stem cells, and most likely are multipotent cells, although they may possibly be pluripotent when they are first recruited to the site.

The invention also refines this study with a method comprising: locating a site comprising an abnormally proliferating cell in a reproductive tissue in a female mammal and contacting the site with a therapeutically effective amount of mammalian extracellular matrix. The contact at the site with the therapeutically effective amount of mammalian extracellular matrix (solid, semi-solid, or liquid extracellular matrix) results in recruiting one or more endogenous stem cells to the site. Sometime after the stem cells have been recruited and the tissue surrounding the damage has begun to remodel, detection of inhibition of the abnormally proliferating cell or cells can be observed, in the presence of the recruited stem cells. The recruited stem cells may be direct actors in the inhibition process, or they may be indirect actors by providing the proper healthy environment including molecular signaling and tissue remodeling for the inhibition to occur eliminating the abnormally proliferating cells. Either mechanism yields a positive result for the patient. Detection of the inhibition of the abnormally proliferating cells can be accomplished by standard visualization techniques (e.g. MRI, CT, PET scans) or by sampling blood marker levels that can indicate a presence or absence of a malignancy, or a reduced amount of malignancy.

Similarly, a method of directing differentiation of a poorly differentiated epithelial cell is accomplished by locating a poorly differentiated epithelial cell in reproductive epithelial tissue in a female mammal, contacting the poorly differentiated epithelial cell with a composition comprising mammalian extracellular matrix, recruiting an endogenous stem cell to the epithelial tissue in the mammal, and observing differentiation of the poorly differentiated cell in the epithelial tissue. As discussed earlier, one of the hallmarks of malignancy is the loss of differentiation of the malignant cell that becomes more and more serious as the cell moves from pre-malignant to fully malignant, losing almost all resemblance it once had to a normal healthy cell. In the presence of recruited stem cells at a site, poorly differentiated cells will become gradually better differentiated as they respond to the local signals generated from the recruited stem cells. New tissue remodels and the once poorly differentiated cells become closer and closer to normal clearly differentiated cells in appearance.

The concentration of the liquid or semi-solid extracellular matrix used in any of these methods is greater than about 0.001 mg/ml. Optimally, the concentration is that concentration that minimally will regenerate missing tissue, heal damaged tissue, inhibit abnormal cell proliferation, and prevent tumor recurrence. This concentration is expected to be at least 10 mg/ml, and as much as 40 mg/ml, or greater. The larger the concentration of matrix, the more effective the healing of the tissue at the site, and so at concentrations greater than 40 mg/ml the composition are expected to be optimally therapeutically effective at healing the site, and inhibiting proliferation of abnormally proliferating cells, and inhibiting the reforming of tumor cells at a site of tumor resection. Furthermore, the exogenous extracellular matrix at the site heals the damaged tissue with reduced or absent scar formation. Solid forms of extracellular matrix can be used, including particulate and sheets as well as any other solid form. The particulate can be dusted in a region, the sheet can be affixed to tissue to heal, for example skin tissue in the case of skin cancer, or the outer layer of an internal organ.

The contact made between the extracellular matrix and the abnormally proliferating cell is made in vivo, in the person or animal afflicted with the abnormal cell proliferation disorder. The contact can be made with the abnormally proliferating cell in the tissue that the cell is growing. For example, a surgeon can identify a tumor in a patient and contact the tumor with sufficient extracellular matrix to cover and surround the tumor or aggregate of cancer cells. Alternatively, a group of such abnormally proliferating cells can be removed and the extracellular matrix composition can be applied at the site of tumor removal, to prevent tumor recurrence. Thus the extracellular matrix composition can be applied to tissue after surgical resection of a tumor, before closing the site. For this reason, subcutaneous tumor models are not ideal in which to demonstrate the full scope and potential of the invention. The contact of exogenous extracellular matrix needs to be made not only with the abnormally proliferating cells, but also with the tissue (and extracellular matrix) that the abnormally proliferating cells are proliferating in. This is because the work that the exogenous extracellular matrix can facilitate is directly tied to its ability to set straight and make right the environment that the malignant cells are propagating in. Subcutaneous tumors have cancer cells growing in clumps virtually unconnected to a tissue, and never connected to the tissue type that that the cells came from. Thus that artificial scenario does not allow the exogenous extracellular matrix to accomplish its full work, that of both remodeling tissue at a site of damage and affecting and influencing the course of the malignancy in addition, or as a result.

To accomplish good contact with the tissue at the site, emulsified, injectable, foam, gel, liquid, glue, paste, small piece, patch, strip, pellet, plug, strand, weave, spray, paint, cream or any malleable, tissue-attachable form of the extracellular matrix material can be used. Conceivably, small pieces, small patches, plugs, strips, pellets, or strands of extracellular matrix material can be attached at the site, or placed there and will work to regenerate healthy tissue at the site of tumor resection. In general, any malleable or appropriate tissue-attachable form of extracellular matrix material that can also contact the abnormally growing cells attached to the tissue can be used at the site, the form primarily being determined by the nature of the tissue, the nature of the cancer cells being targeted for growth inhibition, the nature of the resection, and the anticipated needs for healing and tissue regeneration at the site. For example, sheets of extracellular matrix can be used to close wounds at the dermis with a melanoma or other skin cancers. Optimal compositions of extracellular matrix are liquid, semi-solid or solid formulations.

The extracellular matrix can be in a fluid or liquid form, for example an emulsion or otherwise injectable solution. The extracellular matrix can also be in a semi-solid form, for example a gel, foam, glue, paste, or other semi-solid form. The semi-solid forms may be injectable depending on their viscosity, but they should be applicable to the abnormally proliferating cells or the resected tumor space.

The extracellular matrix can also be a solid, for example any solid form including a powder or particulate that can be sprayed or dusted in a region. The extracellular matrix contacts the tissue and is applied to the site as emulsified, injectable, foam, gel, liquid, glue, paste, small piece, patch, strip, pellet, plug, strand, weave, spray, paint, cream and any malleable form. The extracellular matrix can be a sheet to repair an epithelial layer. Depending on the nature of the tissue and site small patches, plugs, strips, pellets, strands or some other such similar fragments or pieces of solid or semi-solid extracellular matrix material can be used effectively at certain sites with certain tissues, depending largely on the architecture of the tissue, and considering how best to introduce the extracellular matrix. The preferred form of the extracellular matrix will be that form, either liquid, semi-solid or solid that provides maximal contact of the matrix with the tissue or cells that are targeted. So, for example, a solution or emulsion or gel of extracellular matrix will fill a closed space and provide ample contact of the matrix with the tissue of the surrounding region.

Where a tumor has been identified in the patient, the tumor can be addressed by contacting the tumor with an aliquot of extracellular matrix sufficient to cover the tumor and contact all the abnormally growing cells possible. The tumor may also be resected and the space left by the removed tumor can be filled with extracellular matrix. The tumor growth can include, for example, any tumor stage. The composition can also be placed at a site of abnormal cell growth, which can cause an interruption of the abnormal cell growth, and a remodeling of the unhealthy tissue to new healthy tissue. Placing extracellular matrix at a site having cancer or precancer cells can serve to eliminate the cancer cells from the local tissue environment and so eliminate their potential for tumor formation in the body. Placing extracellular matrix in the tumor space can ensure that the tumor will not recur at the site of removal, and will additionally help to heal the tissue so that scarring and disfigurement is limited.

Means of placing the extracellular matrix at the site in the body where a tumor has been resected, or cancer cells are believed to exist, can be accomplished by surgically opening the site and accessing the tissue directly, or by percutanous or other minimally invasive access to the site of cancer or the site where cancer is believed to be. Direct injection of the extracellular matrix at the site either of abnormal cell proliferation or tumor resection may be preferred. A catheter may be able to resect the tumor or lesion of abnormal cells and afterwards apply a coating of extracellular matrix in an injectable, emulsion or spray, for example. Solid pieces of extracellular matrix can be sutured, stapled, glued, or otherwise attached at a site. Plugs, pellets or other pieces of extracellular matrix can be placed or attached at the site using an appropriate attachment means, such a glue or suture.

Detection of developing carcinoma before tumor excision, recurring carcinoma after excision, or forming scar tissue (or lack thereof) after tumor excision followed by placement of ECM at the site of tumor excision, or tissue regeneration can be accomplished by a novel technology called Magnetic Resonance Elastography (MRE). MRE can be used to determine the stiffness of inelasticity of a particular tissue. Where, for example, fat tissue has a kPa of 4, and healthy tissue has a kPa of 6, cancer (carcinoma) will often have a kPa in the realm of a value of 20. Scar tissue may have a kPa between that of healthy tissue and cancer, or perhaps a value stiffer than that of cancer. The absolute value of various scar tissues in the various tissues and organs of interest for the purposes of this invention can be determined by routine application of MRE to these tissues or organs in patients both having and not having carcinoma or scarring. Basic information on the technology called MRE can be found at Doyley et al. “Thresholds for detecting and characterizing focal lesions using steady-state MR elastography,” Medical Physics, 30(4):495-504, 2003.

The compositions of the invention are pharmaceutical compositions which mean that they are acceptable for administration in humans and meet the standards required by the FDA. Any exipient used to make the composition, as with the liquid or semi-solid pharmaceutical compositions are made using pharmaceutically acceptable exipients so that the final composition is suitable, safe and effective for use in humans. The pharmaceutical compositions include an extracellular matrix component, and may also include derivatives or active agents related to it, and also possibly additional pharmaceutical agents that complement, or otherwise contribute to the function of the composition as a whole, such as a carrier, exipient, anti-cancer drug or the like.

By an “effective” amount or a “therapeutically effective amount” of a pharmacologically active agent such as the extracellular matrix it is meant that a nontoxic but sufficient amount of the agent is used in the composition to provide the desired effect of facilitating growth inhibition of abnormally proliferating cells, healing of wounded tissue, or inhibition of tumor recurrence.

### EXAMPLES

Using mice that develop ovarian cancer in a natural and genetically modified model, the mice develop ovarian cancer and are surgically treated for ovarian cancer by removing visible tumors in the reproductive space. The proposal is to then lavage the peritoneal cavity with an emulsion or gel of extracellular matrix to coat as many surfaces as possible before closing the animal. We may also test dusting the entire region with dry particulate of extracellular matrix. The animal will then be monitored for tumor recurrence. Control animals may be surgically treated animals who are not lavaged with extracellular matrix material.

Parameters that may be tinkered with in this system are the concentration of protein in the gel or emulsion. In conjunction with or instead of lavage, the space should be sprayed with dry extracellular matrix particulate so that the matrix material contacts and sticks to the tissue being protected. At sites of tumor removal, solid pieces of extracellular matrix may be placed, such as plugs, strips or other sold pieces.

It is estimated that such a treatment would be successful in a human if the extracellular matrix material is able to inhibit attachment of tumor cells for up to 4 days after surgery. After 4 days, new cancer cells that fail to attach to tissue fall away and are sluffed by the body.

All references cited are incorporated in their entirety. Although the foregoing invention has been described in detail for purposes of clarity of understanding, it will be obvious that certain modifications may be practiced within the scope of the appended claims.

What is claimed is:

1. A method of treating a patient having ovarian carcinoma comprising:
a) identifying an ovarian carcinoma in a female patient,
b) surgically removing said carcinoma from said patient, and
c) contacting a peritoneum and tissues proximal to said removed ovarian carcinoma with a therapeutically effective amount of exogenous mammalian extracellular matrix.

2. The method of claim 1, further comprising surgically closing said patient.

3. The method of claim 1, further comprising monitoring said patient for wound healing.

4. The method of claim 1, further comprising monitoring said patient for recurrence of said carcinoma.

5. The method of claim 1, further comprising monitoring said patient for tissue regeneration.

6. The method of claim 1, further comprising monitoring said patient for scar tissue formation.

7. The method of claim 1, wherein said extracellular matrix is in a form selected from the group consisting of a solid, a semi-solid, and a liquid.

8. The method of claim 1, wherein said extracellular matrix is porcine, bovine, or human.

9. The method of claim 1, wherein said extracellular matrix is small intestine submucosa, liver basement membrane, urinary bladder submucosa, or stomach submucosa.

10. The method of claim 1, wherein said extracellular matrix is a particulate, emulsion, gel or sheet.

11. A method of treating a female patient having a reproductive carcinoma located in a peritoneum of said patient comprising,
a) identifying said reproductive carcinoma in said female patient,
b) surgically removing said carcinoma from said patient, and
c) contacting a peritoneum and tissues proximal to said removed carcinoma with a therapeutically effective amount of exogenous mammalian extracellular matrix.

12. The method of claim 11, wherein said carcinoma is selected from the group consisting of ovarian, uterine, and follicular.

13. The method of claim 11, further comprising monitoring said patient for wound healing, scar tissue formation, or tissue regeneration.

14. The method of claim 11, further comprising monitoring said patient for recurrence of said carcinoma.

15. The method of claim 11, wherein said extracellular matrix is semi-solid or liquid in a concentration greater than about 0.001 ng/ml.

16. The method of claim 11, wherein said extracellular matrix is porcine, bovine, or human.

17. The method of claim 11, wherein said extracellular matrix is small intestine submucosa, liver basement membrane, urinary bladder submucosa, or stomach submucosa.

18. The method of claim 11, wherein said extracellular matrix is a particulate, emulsion, gel or sheet.

19. A method comprising:
a) detecting an ovarian tumor in a patient
b) accessing a peritoneal space comprising said tumor using a minimally invasive surgical device,
c) ravaging said peritoneal space comprising said tumor comprising said tumor with a composition comprising exogenous mammalian extracellular matrix.

20. The method of claim 19, wherein said exogenous mammalian extracellular matrix is selected from the group consisting of particulate, emulsion, gel, a sheet, porcine, bovine, human, small intestine submucosa, urinary bladder submucosa, liver basement membrane, and stomach submucosa.
